(19) United States
(12) Patent Application Publication
(10) Pub. No.: US 2007/0225725 A1
(43) Pub. Date: Sep. 27, 2007

(54) Modular acetabular component inserter

(75) Inventors: Jackson R. Heavener, Warsaw, IN (US); James Collins, Fort Wayne, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/385,569

(22) Filed: Mar. 21, 2006

(51) Int. Cl. A61F 2/00

(52) U.S. Cl. 606/91

(57) ABSTRACT

A modular acetabular component inserter and method are provided for inserting an acetabular component into a surgical site. The modular acetabular component inserter includes a body defining an acetabular component engagement portion and a handle. Various adapters are mountable to the engagement portion to convert the inserter for use with a corresponding acetabular component.

### FIELD OF THE INVENTION

The present invention relates to instruments for use in surgical procedures. More particularly, the present invention relates to instruments for inserting acetabular implants during hip replacement surgery.

### BACKGROUND

Total hip arthroplasty is often used to restore function to a diseased or injured hip joint. Positions and directions relative to the hip joint may be described in terms of proximal being nearer the hip joint, distal being further from the hip joint, anterior being nearer the front of the body, posterior being nearer the back of the body, medial being nearer the centerline of the body, and lateral being further from the center line of the body. In total hip arthroplasty, the surfaces of the femur and pelvis are cut away and replaced with substitute implants. In a typical case, the implants include a hip stem component, a femoral head component, an acetabular component, and bone cement.

The femoral bone is prepared by reaming the femoral canal down into the bone along an axis from a proximal position near the hip joint at the upper end of the femur toward a distal position nearer the knee joint at the lower end of the femur. The pelvis is prepared by reaming the acetabulum. The implants may be placed directly in contact with the prepared bone surfaces for bony fixation of the implant. Alternatively, bone cement may be introduced into the prepared canal and acetabulum so that it hardens around and locks the components in place.

The acetabular component is typically provided as a modular device having an outer shell that is anchored in the reamed acetabulum and a bearing insert that is placed in the outer shell. The shell and bearing insert may be provided in different materials, sizes, and shapes to accommodate differing patient needs. Typically, the shell is inserted into and anchored in the acetabulum first and then the bearing insert is placed in the shell. During insertion of the acetabular shell, it is important to orient the shell in the correct medial/lateral and anterior/posterior angles and to engage it firmly with the prepared acetabulum. Various inserters have been proposed for gripping the shell and gauging its orientation. One problem with earlier devices is that each inserter was engageable with only one style of acetabular shell. Each different style of acetabular shell thus required a new inserter designed specifically for that shell.

A recent development in total hip arthroplasty is the use of minimally invasive surgical techniques in which the bone is prepared and the implants inserted through small incisions that cause less trauma to surrounding muscles and other soft tissues such that the patient's recovery is faster. Such minimally invasive surgical techniques can be challenging due to the difficulty in visualizing the surgical cavity and maneuvering the instruments and implants within the tight confines of the incision.

Another recent development is the use of surgical navigation systems in which sensors detect tracking elements attached in known relationship to an object in the surgical suite such as a surgical instrument, implant, or patient body part. The sensor information is fed to a computer that then triangulates the position of the tracking elements within the surgical navigation system coordinate system. Thus, the computer can resolve the position and orientation of the object and display the position and orientation for surgeon guidance. For example, the position and orientation can be shown superimposed on an image of the patient's anatomy obtained via X-ray, CT scan, ultrasound, or other imaging technology.

### SUMMARY

The present invention provides a modular acetabular component inserter for inserting an acetabular component into a surgical site

In one aspect of the invention, the modular acetabular component inserter includes a body defining an acetabular component engagement portion and a handle. Various adapters are mountable to the engagement portion to convert the inserter for use with a corresponding acetabular component.

In another aspect of the invention, the modular acetabular component inserter includes an elongated shaft defining a handle at a first end and an acetabular component engagement end at a second end opposite the handle. A plurality of modular caps and modular threaded inserts are provided. Each of the modular caps is engageable with an acetabular component in axial force transmitting relationship. Each of the modular threaded inserts is threadingly engageable with an acetabular component to couple the acetabular component and modular cap to the inserter shaft such that mounting one of the plurality of caps and one of the plurality of threaded inserts converts the inserter for use with a corresponding acetabular component.

In another aspect of the invention, a method for inserting an acetabular component into a surgical site includes selecting an adapter from a plurality of adapters; engaging the selected adapter with an inserter shaft to adapt the inserter shaft for engagement with a particular acetabular component; engaging an acetabular component with the adapter; and inserting the acetabular component into the surgical site.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of a modular acetabular component inserter according to the present invention showing the inserter with one arrangement of modular components connected to it;

FIG. 2 is an exploded perspective view of the modular acetabular component inserter of FIG. 1 showing modular caps and inserts engageable with the proximal end of the shaft;

FIG. 3 is a partial cross sectional view taken along line 3-3 of FIG. 2;

FIG. 4 is an exploded perspective view of the modular acetabular component inserter of FIG. 1 showing modular alignment devices mountable on the inserter; and

FIG. 5 is a partial cross sectional view taken along line 5-5 of FIG. 1.

### DESCRIPTION OF THE ILLUSTRATIVE EXAMPLES

Embodiments of a modular acetabular component inserter include an acetabular component engagement end and a plurality of adapters mountable to the engagement end. The adapters are interchangeable to convert the inserter for engagement with different sizes and/or styles of acetabular components. The inserter may be used as a universal inserter adaptable to fit a variety of acetabular components. As new sizes and/or styles of acetabular components become available, corresponding adapters may be provided for use with the inserter to adapt it to the new acetabular components without the need to replace the entire inserter assembly. Acetabular components for use with the inserter may include unitary acetabular components, modular acetabular components with a shell and a bearing insert, and/or other styles of acetabular components. The inserter may be used to insert, align, navigate, impact, and/or otherwise manipulate the acetabular component.

For example, the inserter adapters may include modular caps mountable to the engagement end of the inserter. The caps may be provided in different shapes and/or sizes to engage differently shaped acetabular components. The caps may engage the acetabular component in axial force transmitting relationship to transmit insertion forces from the inserter to the acetabular component. The caps may be provided in different shapes and/or sizes to engage a particular acetabular component in different regions such as internally at the dome (polar region), at the rim (equatorial region), at an intermediate region between the dome and rim, on the exterior of the component, and/or any combination of these regions. The ability to engage different regions of the acetabular component permits different impaction techniques such as rim impaction, dome impaction, and/or intermediate impaction to focus the impaction energy on the desired region of the acetabular component for strength reasons and/or surgeon preference. For example, the inserter may be provided with one cap engageable with the dome of an acetabular component and another cap engageable with the rim of the acetabular component. The adapters may have an inserter engaging portion that is common for all adapters and an acetabular component engaging portion that varies to accommodate different acetabular components. The adapters may include various mechanisms for engaging the acetabular component including abutting engagement, press fit engagement, snap fit engagement, clamping engagement, and/or other suitable engagements. Likewise, the engagement mechanism may engage the acetabular component interior and/or the acetabular component exterior via frictional and/or positive engagements. The engagement mechanism may engage a surface, hole, notch, groove, and/or other suitable feature located at the component pole, equator, and/or any intermediate position. Adapter caps may form a sufficient engagement with the acetabular component and inserter shaft to be used alone or they may be used with a supplemental fixation member such as the threaded inserts described below.

The inserter adapters may include threaded inserts that threadingly engage the acetabular component. The threaded inserts may couple the acetabular component directly to the engagement end of the inserter and/or they may be used with modular caps to couple an acetabular component, cap, and inserter assembly. For example, a threaded insert may be engaged with the acetabular component and rotated to draw the acetabular component into tight abutting relationship to a modular cap. The threaded inserts may be provided in a variety of lengths, diameters, thread style, and/or other parameters to engage different acetabular components and/or acetabular component and cap combinations. The threaded insert may have a male thread that engages a female thread of the acetabular component. Alternatively, the threaded insert may have a female thread that engages a male thread of the acetabular component.

The adapters may permit the acetabular component to rotate freely relative to the inserter. Alternatively, the adapters may include antirotation features that permit rotational alignment of the acetabular component during insertion. The antirotation feature may include frictional and/or positive engagement features. For example, the adapters may include a surface, boss, spline, tooth, key, and/or other feature that engages the acetabular component in rotational aligning relationship. The feature may orient the acetabular component in a single predetermined orientation relative to the inserter, it may orient the acetabular component in one of a predetermined plurality of alternative orientations, and/or the acetabular component may be locked in any of an infinite number of rotational positions relative to the inserter.

The adapters may be color coded to aid a user in identifying the appropriate adapter for use with a particular acetabular component.

The acetabular component inserter may include a shaft having a first end defining the engagement end and an opposite end having a handle. The shaft may be straight or curved. For example the shaft may be curved to allow insertion of the acetabular component in a desired orientation with the curved shaft providing clearance for soft tissue, drapes, and/or other obstructions to a straight shaft.

The inserter may include an alignment apparatus to aid in placing the acetabular component in a desired position in the patient's pelvis. The alignment apparatus may include a mechanical alignment mechanism to provide a visual and/or tactile reference for aiding the surgeon in aligning the inserter and acetabular component with an anatomic landmark. The alignment apparatus may be part of a computerized surgical navigation system in which the system tracks the alignment apparatus such that a computer can identify when the acetabular component is placed in a desired orientation relative to the acetabulum. Multiple alignment apparatuses may be used with the inserter. The multiple apparatuses may be simultaneously associated with the inserter and used simultaneously to provide redundant positioning information. The multiple alignment apparatuses may be alternatively releasably connectable to the inserter to permit the inserter to be converted between surgical navigation and mechanical alignment configurations.

A surgical navigation alignment mechanism may include one or more tracking elements detectable by the navigation system. The elements may be detectable acoustically, optically, tactilely, electromagnetically, via radio frequency signals, and/or by other suitable detection arrangements. The tracking elements may be active or passive. Examples of active tracking elements may include light emitting diodes in an imaging system, ultrasonic emitters in an acoustic system, and electromagnetic field emitters in an electromagnetic system. Examples of passive tracking elements may include elements with reflective surfaces.

The surgical navigation system detects the three dimensional position of the tracking elements and resolves the position of the acetabular component. For example, the surgical navigation system may include multiple sensors at known locations that feed tracking element position information to a computer. The computer may then use the position information from the multiple sensors to triangulate the position of each tracking element within the surgical navigation coordinate system. The surgical navigation system can then determine the position and orientation of the inserter and/or acetabular component by detecting the position and orientation of the tracking elements and resolving the position and orientation of the inserter and/or acetabular component from the known relationship between the tracking elements and the inserter and/or acetabular component.

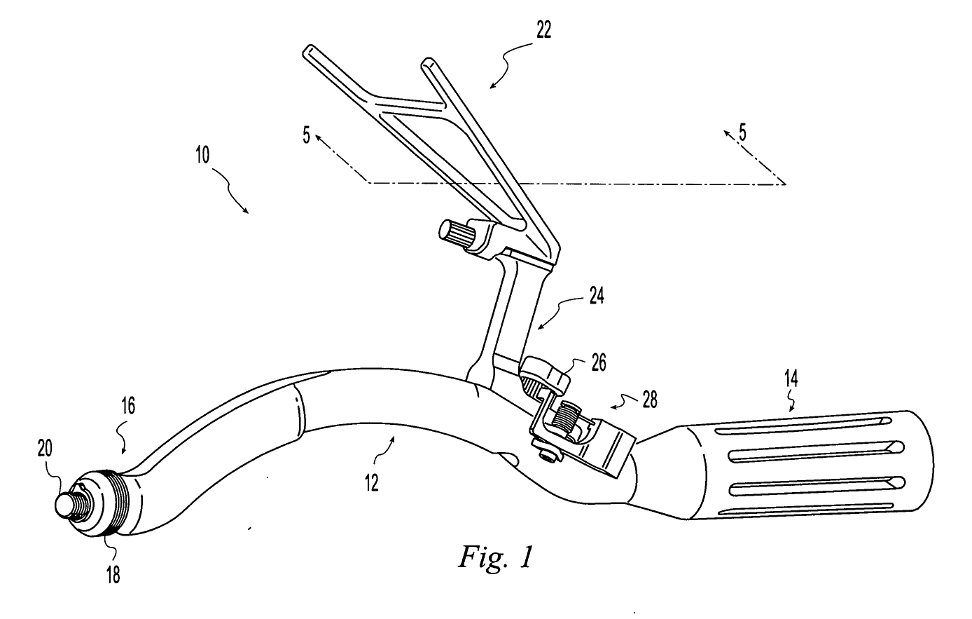

FIGS. 1-5 illustrate an exemplary modular acetabular component inserter 10 according to the present invention. As shown in FIG. 1, the illustrative inserter 10 includes a shaft 12, a handle 14 at one end of the shaft 12, and an acetabular component engagement end 16 at another end of the shaft 12 opposite the handle 14. The engagement end 16 is adaptable to engage different sizes and styles of acetabular components by alternatively mounting a suitable modular cap 18 and/or threaded insert 20. The illustrative shaft 12 is curved to accommodate soft tissues, drapes, and other obstacles near the surgical site. An alignment apparatus 22 is provided to aid in aligning the acetabular component in a desired position at the surgical site. The illustrative alignment apparatus 22 includes a modular assembly permitting its selective use with the inserter 10. The assembly includes a mounting base 24, a locking screw 26, and a ratchet mechanism 28 for preventing the locking screw 26 from unintentionally loosening.

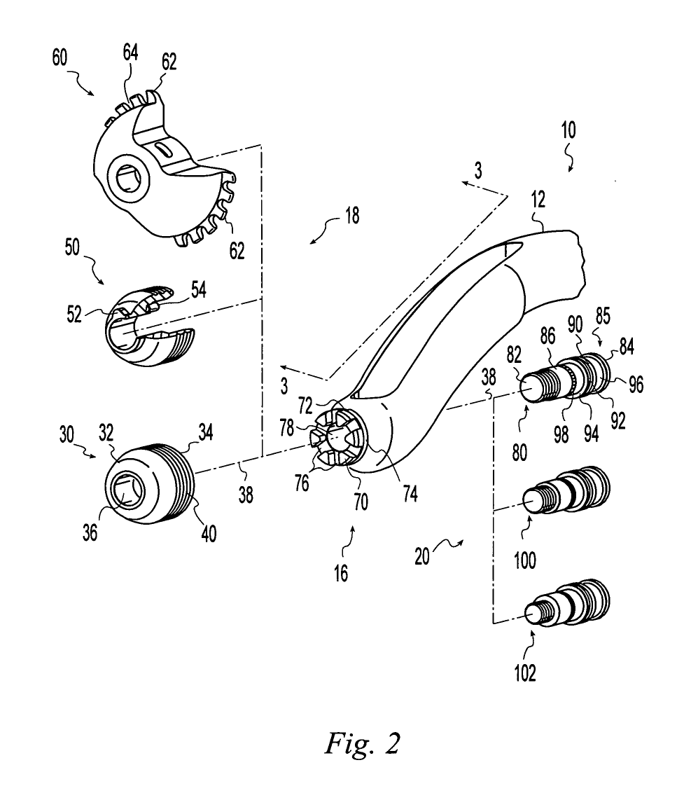

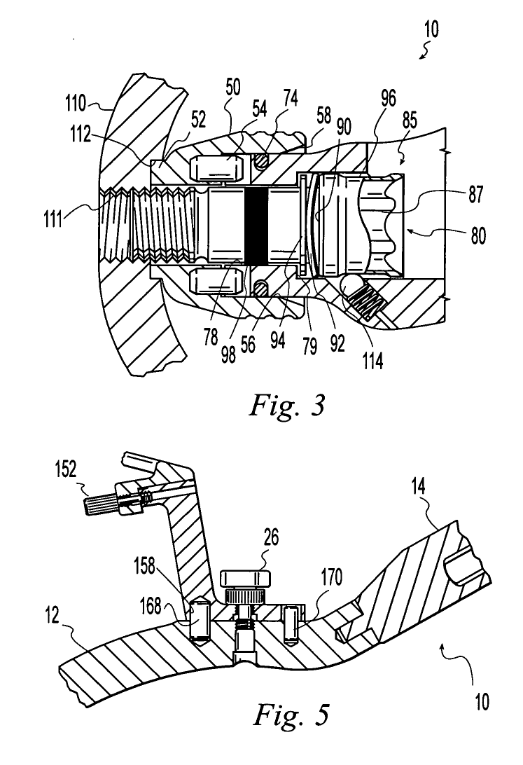

As seen in FIG. 2, the modular cap 18 is provided in different sizes and styles to adapt the inserter 10 for engagement with different sizes and style of acetabular components. A dome bearing cap 30 includes a leading end 32 a trailing end 34, a central longitudinal bore 36, and a longitudinal axis 38. A cavity 56 (FIG. 3) is formed into the cap 30 from the trailing end 34 to receive the engagement end 16 of the inserter 10. The outer surface of the cap includes grooves 40 to facilitate gripping the cap 30. In use, the leading end 32 engages the interior concave portion of a dome shaped acetabular component.

A rotationally keyed dome bearing cap 50 is also shown in FIG. 2. The rotationally keyed cap is similar to the dome bearing cap 30 except that it further includes a lug 52 extending radially along the leading end of the cap 50 to engage the acetabular component in positive rotationally keyed relationship and anti-rotation pins 54 to engage the engagement end 16 of the inserter in positive rotationally keyed relationship. Thus, for example, an asymmetric acetabular component can be rotationally keyed to the inserter to position an asymmetric feature of the component in a desired rotational orientation upon insertion of the acetabular component into the surgical site.

A rim bearing cap 60 includes opposed radially projecting flanges 62 for engaging an equatorial rim of an acetabular component. In the illustrative rim bearing cap 60, the flanges 62 include a plurality of notches 64 for receiving tabs formed on the acetabular component rim. The notches 65 can be selectively engaged with the tabs of the acetabular component in a plurality of alternative rotational positions. Thus, for example, an asymmetric acetabular component can be rotationally keyed to the inserter to position an asymmetric feature of the component in a desired rotational orientation upon insertion of the acetabular component into the surgical site.

The engagement end 16 of the inserter 10 includes a boss 70 projecting from the shaft 12 along an axis 38. The boss 70 is received in the cavity 56 formed in the trailing end of the caps 18. The boss 70 includes a circumferential groove 72 housing a retainer 74. The retainer 74 may be in the form of a split ring, an O-ring, and/or other suitable form. The retainer projects resiliently beyond the groove to frictionally engage the cap 18 as it is inserted over the boss 70 to initially retain the cap 18 on the boss 70. The boss 70 includes notches 76 formed circumferentially around the leading end for rotationally keying the boss to a cap. For example, the rotationally keyed caps 50, 60 include anti-rotation pins 54 that engage the notches to key the caps 50, 60 to the boss 70 as seen in FIG. 3. An axial through hole 78 is formed through the boss 70 and shaft 12 to receive the threaded inserts 20.

Also, as seen in FIG. 2, the threaded inserts 20 are provided in a variety of styles and sizes to adapt the inserter 10 for engagement with different sizes and style of acetabular components. A first exemplary threaded insert 80 includes a leading end 82, a trailing end 84 defining a head 85, a shaft 86 between the leading end 82 and the head 85, and a longitudinal axis 38. The leading end 82 is threaded for engagement with a polar hole in an acetabular component. The thread diameter, pitch, and length may be varied on alternative threaded inserts 100, 102 to engage different acetabular components. Preferably the shaft 86 includes a smooth portion sized for sliding and rotating engagement with the axial through hole 78 in the engagement end 16 of the inserter 10. The head 85 defines a shoulder 90 facing toward the leading end 82. A spring 92 in the form of a Bellville washer is trapped on the shaft 86 between the shoulder 90 and a sliding press fit washer 94. The head 85 defines a circumferential retention groove 96. The illustrative threaded inserts include a color coding band 98 to aid in choosing the correct insert for a particular acetabular component. For example, a colored polymeric compound may be placed in a circumferential groove to form the color coding band 98.

FIG. 3 illustrates how the caps 18 and threaded inserts 20 are assembled to the engagement end 16 of the inserter 10 and the acetabular component 110. The illustrative acetabular component of FIG. 3 is in the form of an acetabular shell including a radial indexing notch 112. The illustrative cap of FIG. 3 is the dome bearing rotationally keyed cap 50 of FIG. 2 and the illustrative threaded insert of FIG. 3 is the first exemplary threaded insert 80 of FIG. 2. In use, a suitable cap and threaded insert are chosen for the acetabular component to be inserted. The cavity 56 formed into the trailing end of the cap 50 is pressed over the engagement end 16 of the inserter in a desired rotational position with the anti-rotation pins 54 engaging the notches 76 in the boss 70. A trailing end chamfer 58 compresses the retainer 74 as the cap 50 is mounted. Frictional engagement between the retainer 74 and the wall of the cavity 56 prevents the cap 50 from inadvertently falling off of the engagement end 16.

The threaded insert 80 is inserted through the hole 78 in the engagement end 16 and boss 70. As the threaded insert 80 is advanced, a ball plunger 114 snaps into the retention groove 96 in the head 85 to help retain the threaded insert in engagement with the inserter 10 so that it does not inadvertently detach from the inserter 10. The retention groove 96 is elongated parallel to the axis 38 so that the threaded insert can slide along the axis 38 while the ball plunger 114 retains the threaded insert 80. The threaded insert 80 is screwed into the polar hole 111 of the acetabular component 110. As the threaded insert 80 is advanced further, the washer 94 abuts an internal shoulder 79 formed in the hole 78 and begins to compress the spring 92 against the shoulder 90 on the insert 80. A wrench may be engaged with a driver socket 87 formed in the head 85 to tighten the threaded insert. Once tightened, the threaded insert couples the acetabular component 110 and cap 50 to the inserter 10. The spring 92 maintains tension on the threaded joint to prevent the threaded insert 80 from vibrating loose if the handle 14 is impacted to seat the acetabular component 110.

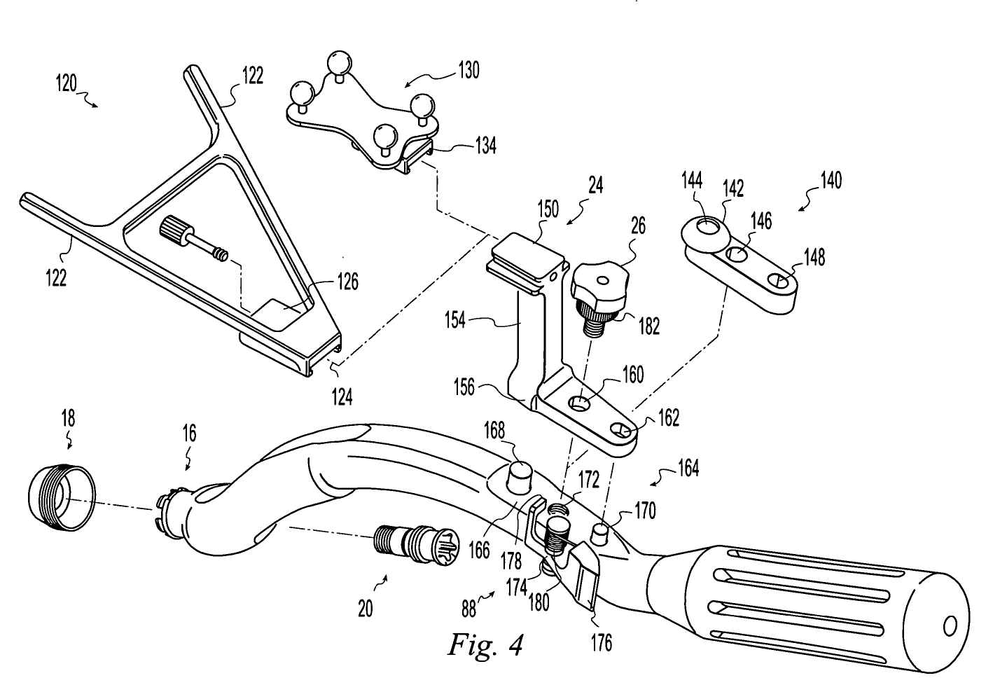

FIG. 4 illustrates alternative alignment apparatuses 120, 130, 140 to aid in placing the acetabular component in a desired position in the patient's pelvis. A mechanical alignment apparatus 120 includes at least one feature visually alignable by a human user with a portion of the patient's anatomy to place the acetabular component 110 in a desired orientation. For example, the illustrative mechanical alignment apparatus 120 includes a pair of arms 122 that angle outwardly from a central axis 124. One arm 122 is used for a left hip and the other is used for a right hip. The arms 122 are further angled upwardly from a dovetail mounting portion 126. By positioning one of the arms 122 parallel to the patient's body, the acetabular component 110 is placed in a desired abduction angle equal to the angle between the arm and the axis 124 and a desired forward flexion angle equal to the angle between the arms and the mounting portion 126.

A first alternative surgical navigation alignment apparatus 130 includes an array of reflective tracking elements 132 trackable by a surgical navigation system and a dovetail mounting portion 134. A second alternative surgical navigation alignment apparatus 140 includes an electromagnetic tracking element 142 trackable by a surgical navigation system and first, second, and third mounting holes 144, 146, and 148.

The mechanical alignment apparatus 120 and the first alternative surgical navigation alignment apparatus 130 may be optionally mounted on a mounting base 24 to elevate the alignment apparatuses above the inserter handle for better visualization by the user and/or the surgical navigation system. The mounting base 24 includes a dovetail slide 150 for engaging the dovetail mounting portion 126, 134 of the alignment apparatuses. A locking screw 152 is threadably engageable with the dovetail slide 150 to retain the alignment apparatuses. A riser post 154 spaces the alignment apparatuses above the inserter handle 14. An attachment base 156 mounts the mounting base to the inserter shaft 12. The attachment base 156 includes a first hole 158 in the base of the riser post 154 (FIG. 5), a second hole 160, and a third hole 162. The inserter shaft 12 includes an alignment apparatus mounting portion 164 including a flat surface 166, a first post 168, a second post 170, and a threaded hole 172 intermediate the first and second posts 168, 170. The first post 168 is received in the first hole 158 of the attachment base 156 and the second post 170 is received in the third hole 162 of the attachment base 156. The locking screw 26 threads through the second hole 160 of the attachment base to removably trap the locking screw 26 on the attachment base 156. In use the mounting base 24 is attached to the inserter shaft 12 by engaging the posts 168, 170 with the first and third holes 158, 162 and threading the locking screw 26 into the threaded hole 172 in the shaft 12.

The inserter 10 includes a ratchet mechanism 28 to prevent the locking screw 26 from loosening during use. The ratchet mechanism 28 includes a lever 174 rotatably mounted to the inserter shaft 12. One end of the lever 174 defines an actuator 176 and an opposite end defines a pawl 178. A spring 180 biases the pawl inwardly toward the locking screw 26 to engage circumferential teeth 182 formed on the locking screw 26. The actuator 176 is pressed inwardly toward the shaft 12, such as by thumb pressure, to pivot the pawl 178 away from the locking screw 26 to allow the locking screw 26 to be loosened.

The second alternative surgical navigation alignment apparatus 140 is mountable directly to the alignment apparatus mounting portion 164 by inserting the posts 168, 170 into the first and third mounting holes 144, 148 and inserting the locking screw through the second mounting hole 146.

In use, one of the modular alignment apparatuses is attached to the inserter shaft (if desired) and locked into place. An appropriate cap and/or threaded insert is chosen to convert the inserter for use with the particular acetabular component to be inserted. The acetabular component is engaged with the inserter and inserted into the surgical site. The positioning of the acetabular component is guided with the alignment apparatus if desired.

Although examples of a modular acetabular component inserter and its use have been described and illustrated in detail, it is to be understood that the same is intended by way of illustration and example only and is not to be taken by way of limitation. The invention has been illustrated in use with an acetabular component in the form of a shell for retaining a modular bearing insert. However, the modular acetabular component inserter may be configured for use with bearing inserts, monoblock acetabular components, and other types of acetabular components. Accordingly, variations in and modifications to the modular acetabular component inserter and its use will be apparent to those of ordinary skill in the art, and the following claims are intended to cover all such modifications and equivalents.

What is claimed is:

1. A modular acetabular component inserter for inserting an acetabular component into a surgical site, the modular acetabular component inserter comprising:
a body defining an acetabular component engagement portion and a handle; and
a plurality of adapters mountable to the engagement portion, each of the adapters being shaped to engage a differently shaped acetabular component such that mounting one of the plurality of adapters converts the inserter for use with the corresponding acetabular component.

2. The modular acetabular component inserter of claim 1 wherein the plurality of adapters comprises a plurality of modular caps alternatively mountable to the engagement portion.

3. The modular acetabular component inserter of claim 2 wherein at least one of the plurality of modular caps comprises a dome shaped portion engageable with a concave interior portion of a domed shaped acetabular component.

4. The modular acetabular component inserter of claim 2 wherein at least one of the plurality of modular caps includes a radially projecting flange engageable with a circumferential rim of a dome shaped acetabular component.

5. The modular acetabular component inserter of claim 1 wherein the plurality of adapters comprises a plurality of modular threaded inserts threadingly engageable with an acetabular component.

6. The modular acetabular component inserter of claim 5 wherein the plurality of threaded inserts are provided in a variety of lengths, diameters, and thread styles.

7. The modular acetabular component inserter of claim 1 wherein the plurality of adapters comprises a plurality of modular caps and a plurality of modular threaded inserts, each of the modular caps being engageable with an acetabular component in axial force transmitting relationship and each of the threaded inserts being threadingly engageable with an acetabular component to couple the acetabular component and modular cap to the inserter body.

8. The modular acetabular component inserter of claim 1 wherein at least one of the plurality of adapters engages both the acetabular component and the body of the inserter in rotationally fixed relationship to permit rotational alignment of the acetabular component during insertion.

9. The modular acetabular component inserter of claim 1 wherein the adapters comprise a color code readable to distinguish one adapter from another.

10. The modular acetabular component inserter of claim 1 further comprising an alignment apparatus to aid in placing the acetabular component in a desired position in the surgical site.

11. The modular acetabular component inserter of claim 10 comprising a plurality of alternative alignment apparatuses engageable with the inserter body.

12. The modular acetabular component inserter of claim 11 wherein the plurality of alternative alignment apparatuses includes at least one mechanical alignment apparatus having a visual alignment reference and at least one surgical navigation alignment apparatus trackable by a computerized surgical navigation system.

13. The modular acetabular component inserter of claim 12 wherein the plurality of alignment apparatuses are alternatively releasably connectable to the inserter body to convert the inserter between surgical navigation and mechanical alignment configurations.

14. A modular acetabular component inserter for inserting an acetabular component into a surgical site, the modular acetabular component inserter comprising:
an elongated shaft defining a handle at a first end and an acetabular component engagement end at a second end opposite the handle;
a plurality of modular caps, each of the modular caps being engageable with an acetabular component in axial force transmitting relationship, each of the modular caps being shaped to engage a differently shaped acetabular component; and
a plurality of modular threaded inserts, each of the threaded inserts being threadingly engageable with an acetabular component to couple an acetabular component and modular cap to the inserter shaft, such that mounting one of the plurality of caps and one of the plurality of threaded inserts converts the inserter for use with a corresponding acetabular component.

15. The modular acetabular component inserter of claim 14 wherein at least one of the plurality of modular caps comprises a dome shaped portion engageable with a concave interior portion of a domed shaped acetabular component and at least one of the plurality of modular caps includes a radially projecting flange engageable with a circumferential rim of a dome shaped acetabular component.

16. The modular acetabular component inserter of claim 14 further comprising a plurality of alternative alignment apparatuses engageable with the inserter body to aid in placing the acetabular component in a desired position in the surgical site.

17. The modular acetabular component inserter of claim 16 wherein the plurality of alternative alignment apparatuses includes at least one mechanical alignment apparatus having a visual alignment reference and at least one surgical navigation alignment apparatus trackable by a computerized surgical navigation system.

18. The modular acetabular component inserter of claim 14 wherein the shaft is curved.

19. A method for inserting an acetabular component into a surgical site, the method comprising:
selecting an adapter from a plurality of adapters;
engaging the selected adapter with an inserter shaft to adapt the inserter shaft for engagement with a particular acetabular component;
engaging an acetabular component with the adapter; and
inserting the acetabular component into the surgical site.

20. The method of claim 19 wherein selecting an adapter comprise selecting a cap from a plurality of differently shaped caps and selecting a threaded insert from a plurality of threaded inserts and wherein engaging an acetabular component comprises engaging the cap with the acetabular component in axial force transmitting relationship and threading the threaded insert into the acetabular component to couple the acetabular component, cap, and shaft together.
